(19) United States
(12) Patent Application Publication
(10) Pub. No.: US 2013/0116530 A1
(43) Pub. Date: May 9, 2013

(54) PROCESS FOR MANUFACTURING AN ELECTRODE FOR MEDICAL USE, AND ELECTRODE OBTAINED

(71) Applicant: Jean-Pierre Darnis, Cugney (FR)

(72) Inventor: Jean-Pierre Darnis, Cugney (FR)

(73) Assignee: DIXI MICROTECHNIQUES, Besancon Cedex (FR)

(21) Appl. No.: 13/810,711

(22) PCT Filed: Jun. 30, 2011
(86) PCT No.: PCT/FR2011/000383

§ 371 Date: Jan. 17, 2013

(30) Foreign Application Priority Data
Jul. 22, 2010 (FR) 1056014

(51) Int. Cl. H01B 13/00; A61N 1/05; A61B 18/08; A61B 5/0478

(52) U.S. Cl. CPC H01B 13/0036; A61B 5/0478; A61N 1/0529; A61B 18/082
USPC 600/378; 427/2.11

(57) ABSTRACT

A method of manufacturing an electrode for medical use, such as an intracerebral electrode (1) intended for use at brain level, the electrode having the shape of a narrow and elongated rod and including at least one electrical contact pad (2, 4) connected to an electrical conductor intended to be connected to a processing device and/or a recording device. In order to realize the electrode (1), a substrate (10) having a tridimensional shape is used and a metal layer is deposited on a periphery of the substrate (10) by a physical vapor deposition technique through a mask (12) that determines a pattern arranged so as to define at least the electrical contact pad (2, 4).

This application is a National Stage Completion of PCT/FR2011/000383 filed Jun. 30, 2011 which claims priority from French Application Serial No. 10/56014 filed Jul. 22, 2010.

### TECHNICAL SCOPE

The present invention relates to a method for manufacturing an electrode for medical use, such as an intracerebral electrode intended for use at brain level, this electrode having the shape of a narrow and elongated rod carrying at least one electrical contact pad connected to an electrical conductor intended to be connected to a processing and/or recording device.

The present invention also relates to an electrode obtained with said method.

### PRIOR TECHNIQUE

The present invention relates more specifically to the area of intracerebral electrodes. Such instruments are used for carrying out electrophysiological brain explorations, in particular with the aim of locating and characterizing epileptogenic foci of epileptic patients, diagnosing and/or treating tumors, or collecting spontaneous activities. The intracerebral electrodes also allow carrying out stimulations such as functional mapping, or triggering seizures. Another application relates to the realization of thermocoagulations intended for the treatment of epilepsy, at the end of a stereoelectroencephalographic stimulation session.

The intracerebral electrodes are classically made of electrical contacts, insulators, conductors, a jacket and connectors assembled together. Due to the very small dimensions of these different parts, their assembly is particularly tedious, requires a very long time, and generates consequently significant labor costs. Furthermore, the intrinsic quality of the intracerebral electrodes manufactured this way is not totally satisfactory today. It has indeed been noted that the mechanical strength of such an assembly of heterogeneous small-size parts is relatively low and may sometimes be insufficient. On the other hand, another disadvantage lies in the fact that the metallic masses of the electrical contacts are important and generate artefacts that can degrade the quality of the images of the explored or stimulated areas collected by magnetic resonance imaging (MRI).

### DESCRIPTION OF THE INVENTION

The present invention aims to remedy these disadvantages by offering a method for manufacturing an intracerebral electrode allowing both to reduce the manufacturing times and to produce an intracerebral electrode having an increased mechanical strength with reduced metallic masses.

To that purpose, the invention relates to a method of the kind stated in the preamble, characterized in that, to realize said electrode, a substrate having a tridimensional shape is used and a metal layer is deposited on the surface of said substrate by means of a physical vapor deposition technique, through a mask that determines a pattern arranged so as to define at least said electrical contact pad.

According to a variant of this method, said metal deposition is carried out in at least two successive steps and said substrate is turned over between said two steps.

According to another variant of said method, the metal deposition is carried out in one single step using at least two targets.

According to a third implementation variant of this method, said metal deposition is carried out by subjecting said substrate to a rotation.

According to another characteristic of the present method, a stainless steel mask that forms a gripper arranged to clamp said substrate is used.

An additional characteristic is further defined by the fact that the area of the substrate that is not covered by said mask is activated chemically, preferably by subjecting it to an ionic cleaning step carried out by means of a mix of oxygen and argon in the plasma state, and by depositing then a layer of titanium on it.

The metal used for defining said electrical contact pad is advantageously gold.

The invention also relates to an electrode for medical use, obtained by the implementation of the method described previously, such as an intracerebral electrode intended for use at brain level, this electrode having the shape of a narrow and elongated rod including at least one electrical contact pad connected to an electrical conductor intended to be connected to a processing and/or recording device, characterized in that said rod forms a tridimensional substrate around which at least one metal layer is deposited by means of a physical vapor deposition technique, through a mask that determines a pattern arranged so as to define at least said electrical contact pad. Said electrical contact pad is preferably made out of gold.

### BRIEF DESCRIPTION OF THE DRAWINGS

The present invention and its advantages will be better revealed in the following description of an embodiment given as a non limiting example, in reference to the drawings in appendix, in which:

FIG. 1 represents a cross-sectional view of a device for the implementation of the method according to the invention, arranged to carry out the metal deposition by subjecting the substrate to a rotation,

FIG. 2 is a top view of an electrode according to the invention, of which one end includes connection cables arranged to be connected to a connector,

FIG. 3 is a top view of a variant of the electrode according to the invention, in which the connector is integrated,

FIG. 4 is a perspective view of a mask used for the implementation of the method according to the invention, in its opened position, and

FIG. 5 is a side view of the mask of FIG. 4, in its closed position.

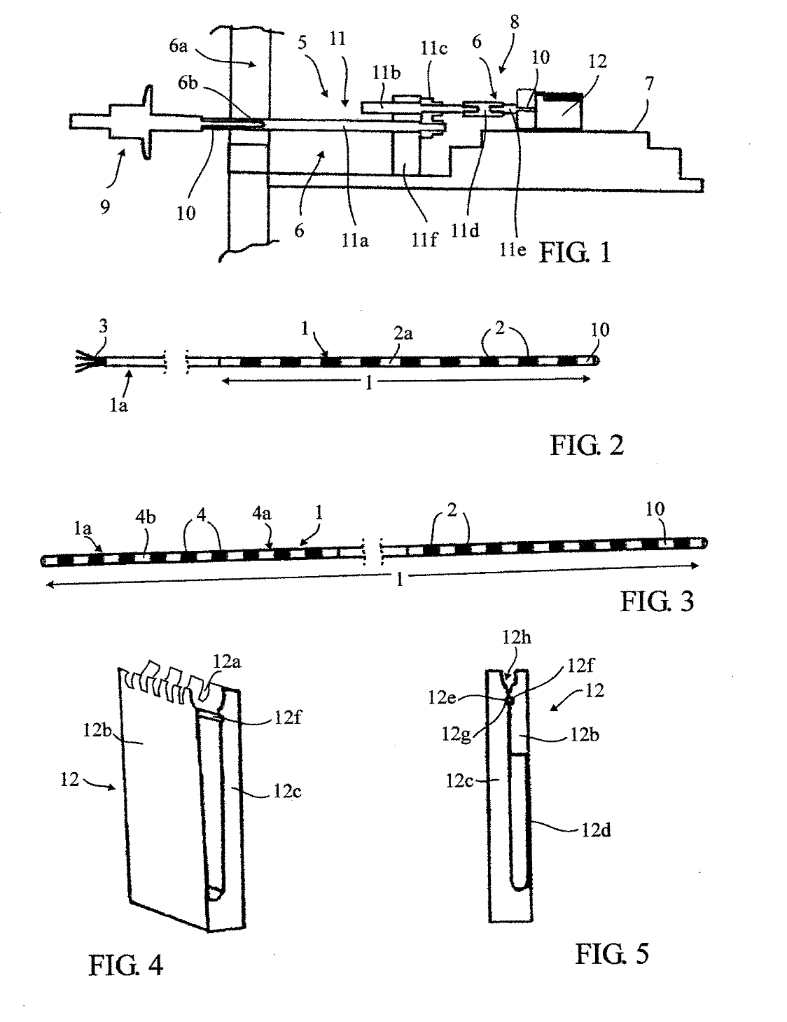

### DETAILED DESCRIPTION OF THE INVENTION

Referring to the figures, the present invention relates to a method for manufacturing an intracerebral electrode 1, having preferably a cylindrical shape and including in a classical way a plurality of electrical contact pads 2, distributed on the periphery and on at least a part of its length l. These electrical contact pads 2 allow performing operations such as, for example, recording brain activity, brain stimulation and/or the realization of previously mentioned thermolesions.

According to an embodiment variant represented in FIG. 2, the contact pads 2 can be connected to a plurality of connection cables 3 extending from an end 1a of electrode 1 and arranged so as to connect said electrode 1 via a non represented connector, for example of the multicontact type, to the medical equipment selected according to the nature of the operations to be carried out at brain level. According to another variant, represented in FIG. 3, electrode 1 comprises advantageously, at the level of its end 1a, a series of contact pads 4 connected electrically to the contact pads 2 of electrode 1 and defining an integrated cylindrical multicontact connector 4a, arranged to be connected directly to said medical equipment.

Such an intracerebral electrode 1 has classically a rigid or semi-rigid structure, with a diameter of about 0.8 mm, and its length l, and consequently the number of contact pads 2, may be variable.

In the represented example, the intracerebral electrode 1 is manufactured from a substrate 10 having a cylindrical shape, which defines the body of the electrode, on the surface of which a layer of metal, preferably gold or any equivalent electrically conductive metal, is deposited by means of a physical vapor deposition technique, according to a pattern that defines the contact pads 2, and, if necessary—the contact pads 4. The substrate 10 used is made of a biocompatible material, suitable for implantation in the brain tissue, this material being for example chosen in the group including polyamide, polyetheretherketone, pebax®, polyimide, reinforced polyimide, polyurethane, Tecoflex® polyurethane, Rilsan®.

In order to ensure a uniform metal deposition on the whole circumference of substrate 10, the present method provides advantageously, in compliance with the illustrated embodiment variant, to subject it to a rotation during all manufacturing steps of electrode 1. This goal is achieved by the implementation of device 5, which includes in a conventional way a vacuum vessel 6 equipped with a physical vapor deposition cell 7, and means 8 for receiving substrate 10 arranged to subject the latter to a rotation inside of said vacuum vessel 6. Said rotation means 8 are controlled from the outside of said vessel 6 and are coupled to that purpose with driving means 9 such as an electrical motor or a similar device (not represented) located outside of said vacuum vessel 6, by means of a driving shaft 11 passing through a wall 6a of vacuum vessel 6, through a sealed bearing 6b. Drive shaft 11 may be coupled directly or indirectly with receiving means 8, as in the represented example. To that purpose, drive shaft 11 comprises a first axis 11a coupled on the one hand with the motor and on the other hand with a second axis 11b via a gear comprising two toothed wheels 11c. Axis 11b itself is coupled, by means of an insulating support 11d, with a third axis 11e connected with an end of substrate 10. Axes 11a, 11b are preferably guided in bearings provided in a support 11f that guarantees their positioning, parallelism and alignment.

In compliance with the present method, depositing the metal layer that defines contact pads 2, 4 occurs after a chemical activation step of the concerned area of said substrate 10. This activation step, carried out for example within device 5, consists in performing an ionic cleaning of said concerned area of substrate 10, for example using a mix of oxygen and argon, followed by the deposition of a layer of titanium.

Advantageously, the steps of activation of substrate 10, then of deposition of the metal layer, are carried out enclosing the free end of substrate 10 inside of a mask 12 having openings 12a that determine the pattern arranged in order to define the electrical contact pads 2, 4. According to an embodiment example illustrated by FIGS. 4 and 5, mask 12 can have the form of a clamp comprising two parallel legs 12b, 12c, connected at their base by means of an elastic tongue 12d that pushes said legs 12b, 12c in their closed position. Such mask 12 comprises a guiding housing 12e (see FIG. 5) that can receive substrate 10, this housing being created by bringing together two grooves 12f, 12g formed on the internal faces of legs 12b, 12c of mask 12. The free ends of legs 12b, 12c form a “V”-shaped introduction area 12h located above guiding housing 12e to allow passing substrate 10 through the opening and the automatic closing of clamp 12, achieved thanks to the V-shape of the introduction area and to the elastic tongue 12d. Guiding housing 12e allows maintaining perfectly the position of substrate 10 with respect to openings 12a during the activation and metal deposition steps. The use of such mask 12 consequently allows carrying out the metal deposition in a very accurate way, while avoiding any unexpected deposition in spaces 2a, 4b that separate two contact pads 2, 4. Such mask 12 is preferably manufactured by machining a part out of stainless steel or any other material having the same flexibility properties by means of an electroerosion cutting method or by any other equivalent manufacturing method.

According to another variant of the present method, implemented by means of a not represented device, it is also possible to perform the metal deposition on the surface of a substrate 10 in at least two successive steps. In this case, it is provided, in a first step, to expose first a first side of substrate 10 to said metal, then to turn over said substrate 10 and to repeat this same operation on another side of said substrate 10.

According to a third variant of the present method, it is also possible to carry out said metal deposition simultaneously on at least two sides of substrate 10, by means of a not represented device comprising at least two targets.

The choice of these various methods will be determined according to the constraints linked with the manufacturing in more or less large series of this type of electrodes.

### POSSIBILITIES FOR INDUSTRIAL APPLICATION

This description shows clearly that the invention allows manufacturing intracerebral electrodes in an automated way, with high accuracy, in series, with an excellent reproducibility, allowing reducing the labor costs. Furthermore, the tests that have been performed allowed to show that the gold layer deposited this way had an excellent surface bonding strength, leading to intracerebral electrodes characterized by a mechanical strength higher than that observed for the classical intracerebral electrodes.

The present invention is not restricted to the example of embodiment described, but extends to any modification and variant which is obvious to a person skilled in the art while remaining within the scope of the protection defined in the attached claims.

1-10. (canceled)

11. A method of manufacturing an electrode for medical use, the electrode being formed in a shape of a narrow and elongated rod comprising at least one electrical contact pad (2, 4) connected to an electrical conductor intended to be connected to at least one of a processing device and recording device,
wherein, in order to realize the electrode (1), a substrate (10) having a cylindrical shape, made from a biocompatible rigid or semi-rigid material, which defines the body of the electrode, is used, and
a metal layer is deposited on the surface of the substrate (10) by a physical vapor deposition technique, through a mask (12) that determines a pattern arranged so as to define at least the electrical contact pad (2, 4).

12. The method according to claim 11, further comprising the step of depositing the metal layer along an entire circumference of the substrate (10),
carrying out the metal deposition in at least two successive steps, and
turning over the substrate (10) between the two successive steps.

13. The method according to claim 11, further comprising the step of depositing the metal layer along an entire circumference of the substrate (10), and
carrying out the metal deposition in one single step using at least two targets.

14. The method according to claim 11, further comprising the step of depositing the metal layer along an entire circumference of the substrate (10), and carrying out the metal deposition by subjecting the substrate (10) to rotation.

15. The method according to claim 11, further comprising the step of using a stainless steel mask (12) that forms a gripper arranged to clamp the substrate (10).

16. The method according to claim 11, further comprising the step of, prior to the deposition of the metal layer, chemically activating an area of the substrate (10), that is not covered by the mask (12), by an ionic cleaning carried out by a mix of oxygen and argon in a plasma state, and
then depositing a layer of titanium is then deposited on the area of the substrate (10), that is not covered by the mask (12).

17. The method according to claim 11, further comprising the step of using gold as the metal for defining the electrical contact pad (2, 4).

18. A method of manufacturing an intracerebral electrode (1) for medical use at brain level, the electrode being formed in a shape of a narrow and elongated rod comprising at least one electrical contact pad (2, 4) connected to an electrical conductor intended to be connected to at least one of a processing device and recording device,
wherein, in order to realize the electrode (1), a substrate (10) having a cylindrical shape, made from a biocompatible rigid or semi-rigid material, which defines the body of the electrode, is used, and
a metal layer is deposited on the surface of the substrate (10) by a physical vapor deposition technique, through a mask (12) that determines a pattern arranged so as to define at least the electrical contact pad (2, 4).

19. An electrode for medical use, the electrode being formed in a shape of a narrow and elongated rod comprising at least one electrical contact pad (2, 4) connected to an electrical conductor intended to be connected to at least one of a processing device and recording device,
wherein the rod being formed as a cylindrical substrate (10) made out of a biocompatible material around which at least one metal layer is deposited by a physical vapor deposition technique, through a mask (12) that determines a pattern arranged so as to define at least the electrical contact pad (2, 4).

20. The electrode for medical use according to claim 8, further comprising the step of using gold as the metal for defining the electrical contact pad (2, 4).
